(19) United States
(12) Patent Application Publication
(10) Pub. No.: US 2023/0080869 A1
(43) Pub. Date: Mar. 16, 2023

(54) Use of an aliphatic ester of a polyglycerol phosphate as an emulsifier for the preparation of compositions for trichological use

(71) Applicant: Greengredients S.r.l., Terni TR (IT)

(72) Inventor: Guido BREGAGLIO, Terni TR (IT)

(21) Appl. No.: 17/797,754

(22) PCT Filed: Feb. 11, 2021
(86) PCT No.: PCT/IB2021/051109

§ 371 Date: Aug. 5, 2022

(30) Foreign Application Priority Data
Feb. 12, 2020 (IT) 102020000002797

(51) Int. Cl. A61K 8/85; A61K 8/37; A61Q 5/12; A61K 8/06

(52) U.S. Cl. CPC A61K 8/85; A61K 8/37; A61Q 5/12; A61K 8/06; A61K 2800/5424; A61K 2800/42

(57) ABSTRACT

This patent application relates to the use of an aliphatic ester of a polyglycerol phosphate as an emulsifier for the preparation of compositions for trichological use, wherein the polyglycerol has a number of monomeric units comprised between 4 and 20; the phosphate ester is a polyglycerol monoester; the aliphatic ester is an polyglycerol ester with one or more fatty acid molecules selected from the group consisting of saturated, monounsaturated and polyunsaturated fatty acids with a number of carbon atoms comprised between 4 and 22 or their ester derivatives.

### SCOPE OF THE INVENTION

This patent application concerns the use of emulsifying agents for the preparation of cosmetic products.

### STATE OF THE ART

Hair colours are used by more than 60% of women. There are many reasons why hair colouring is used: some use it to cover grey hair, others to satisfy a need for change, others to make their hair more vibrant. The average frequency of use is at least 6-8 times a year.

Both professional and home-made hair dyes are increasingly used also by men (10-15%) who dye their hair to mask the signs of ageing or to gain glamour.

Oxidation colouring products (oxidation dyes) are the type of colouring that can lighten or darken the natural colour of the hair, intensify or modify its reflections and guarantee a long-lasting result.

They are generally creamy products, less frequently oily or gelatinous, containing colourless substances which only become true permanent colours after oxidation. The contents of tubes or bottles of dye are not a true colour, but a colour precursor which, following chemical reactions of oxidation and condensation, forms colouring substances.

The colouring mechanism is based on the use of small substances known as precursors. These are molecules which, thanks to their size, penetrate into the hair fibre through the cuticle and reach the cortex.

The use of alkalising agents (such as ammonia or other substances from the amine group) assists the penetration of the colour precursor into the hair by swelling the hair.

The most common oxidising agent used in hair dyeing is hydrogen peroxide (H₂O₂).

Colour precursors are organic compounds belonging to the benzene series in which amine-NH2 or alcohol-OH groups are present. The most commonly used products include para-diaminobenzene, meta-dihydroxybenzene and para-aminophenol.

Once penetrated into the hair shaft, these substances react with the oxygen produced by the decomposition of the hydrogen peroxide that is added to the dye at the time of use, forming larger pigments that remain embedded in the hair fibre.

These coloured pigments have a high covering power and are particularly stable against light and the action of chemical agents such as shampoo, conditioner, hairspray, etc.

Hydrogen peroxide not only allows the formation of pigments but, at the same time as activating the colour precursor, it also decolourises the hair, i.e. it denatures the melanin, its natural pigment. As a result, the hair is bleached and becomes more susceptible to taking on a different colour, even in the case of a lighter shade than its natural colour.

The choice of hydrogen peroxide concentration is determined by the type of colour to be obtained or the percentage of white hair present. Normally, high concentration hydrogen peroxide (30-40% v/v) is intended for decolouring treatments, aimed at bleaching the original colour, while low concentration hydrogen peroxide (10-20% v/v) is more suitable for tone-on-tone colouring treatments.

Oxidising dyes consist of two products that must be mixed before use: the alkaline agent that contains the colour precursors and the oxidising agent that activates the precursor.

The alkaline agent works by opening up the outer layer of the hair, allowing the oxidiser and the colour precursor to penetrate deep into the hair; the alkaline agent bleaches the melanin pigments and activates the colour precursor, which then colours the hair.

Oxidising dyes change the hair's natural pigments, giving permanent colour with a lasting effect that cannot be removed by washing.

For the preparation of cream hair dyes an emulsifying system is used, which is capable of forming a product with the necessary characteristics to perform its task: resistance to mixing with the acid phase of development; stability and modulation of oxidation; adhesion to the hair; ability to develop full, particularly homogeneous and covering shades.

The emulsifying system commonly used in a hair dye cream composition is composed of fatty alcohols from C12 to C22 (e.g. lauryl, myristyl, cetyl, cetylearyl, benyl, etc.) with non-ionic polyoxyethylene emulsifiers (alkyl ethers), anionic emulsifiers (e.g. sulphated alkyl ethers), fatty acid glycerides, waxes, etc.).

The emulsifying system represents 20-35% of the product and is a decisive component in the composition of a hair dye cream.

Hair dye manufacturers base the formulations of the various colours (a ‘colour chart’ is composed of at least 70 shades, some of which are updated every year according to fashion) on the chosen emulsifying system, relying on it for product stability, chemical-physical characteristics (appearance, homogeneity, body, etc.) and rheological properties (viscosity, consistency, application on the hair, etc.).

The manufacturer's work focuses above all on developing the finished product, which must create the required colour, coverage, reflection and shine on the hair.

The world of raw materials for cosmetic use has been in turmoil for some time because factors of environmental concern and the protection of human health are leading to a critical review of the products used up to now, which are potentially harmful to humans and the ecosystem.

The rate of renewal is very high, due to the incessant demand for new products in line with the changing demands of the end consumer, who favours the use of raw materials from renewable sources and produced using processes that respect the environment.

With regard to emulsifiers, research has long been oriented towards the study of alternatives to ethylene oxide derivatives, looking at more natural and safer hydrophilic structures.

### Prior Art Problem

Polyoxyethylenated emulsifiers are still the most widely used emulsifiers in the hair sector, as they are simple, safe and economical to use. The polyoxyethylenate category is the most widely used class of emulsifiers in chemical products in general (household and car products, industrial products, etc.) and all their characteristics and uses are well known. However, these emulsifiers have limited biodegradability, poor tolerability and dermal compatibility, as well as the unfavourable connotation of being ‘chemically synthesised products’.

The substitution of polyoxyethylenates by alternative emulsifiers is a complex and difficult operation, leading to significant product instability problems.

In cosmetic formulations for personal care and hygiene, the use of alternative (‘green’ or ‘new generation’) emulsifiers is becoming more and more popular, since the chemical environment in which the emulsifier is placed is characterised by mild pH conditions (neutral), or is free of destabilising ingredients, such as strong acids and bases, solvents, electrolytes, etc.

Trichological compositions, such as oxidation dyes, decolouring products, masks for conditioning, hair reconstruction or finishing (curling, straightening, anti-frizz, etc.) are characterised by ingredients that are difficult to emulsify, as well as by extreme pH conditions, due to the presence of alkalising or acidic agents.

For this reason, it is complicated to formulate trichological products, including alternative emulsifiers, that meet, on the one hand, the stability, compatibility and performance requirements of the market and, on the other hand, the requirements for the recognition of standard certifications for organic and natural cosmetics (e.g. Cosmos, Icea, Ecocert, Bdih, Natrue etc.), which are increasingly necessary to face the market.

The Applicant has experimented with PEG-free emulsifiers (free of polyethylene glycols) belonging to the chemical classes available on the market (e.g. polyglycerol esters or ethers with fatty acids, sorbitol esters with fatty acids, fatty alcohol esters with carboxylic/bicarboxylic acids or with phosphoric acids, acylglutamates, sucroesters, alkylpolyglucosides, protein derivatives and/or lipoamino acids etc.), but the experiments carried out have not produced satisfactory results.

### SUMMARY OF INVENTION

The Applicant has now found that it is possible to solve prior art problems by employing phosphate polyglycerols, esterified with aliphatic fatty acids or their ester derivatives, as an emulsifying base for the preparation of compositions for professional or home trichological use.

The specified technical task and purposes are substantially achieved by the technical characteristics set forth in one or more of the claims.

### Advantages of the Invention

The esterified polyglycerol phosphate is suitable for forming a trichological emulsifying composition satisfactory in terms of stability, performance and safety of use. In addition to having the emulsifying characteristics that ensure the stability of the products, the use of an aliphatic ester of a polyglycerol phosphate for the preparation of trichological compositions has the following advantages:
It helps the dispersion of colour pigments and contributes to the formation of homogeneous products;
It can be used in permanent, semi-permanent and temporary colouring products;
It improves the rate at which colour is deposited in the hair and makes it possible to achieve unexpected brilliance results, as well as colour intensity and homogeneity;
In particularly aggressive technical formulations (e.g. permanent colouring, decolouration, formulations that cause oxidative damage to the hair, alter the structure and physiological pH of the hair) it makes it possible to obtain full-length hair that is easy to comb and condition;
It can be used indifferently in hair dye formulations based on the action of ammonia or amines (ammonia-free dyes);
It can be used in hair dye formulations containing both paraphenylenediamine and paratoluenediamine, which are the two most common colour precursors on the market.

The aliphatic ester of polyglycerol phosphate can advantageously be used in the same percentages as traditional emulsifier systems containing polyethylene glycols (PEG). The achievement of colour tones and various nuances is optimal, making it possible to achieve results that sometimes cannot even be achieved with normal carrier bases (based on PEG), which often fail to graduate the development of active oxygen in the application time.

### DESCRIPTION OF FIGURES

FIG. 1: Schematic representation of the process for the synthesis of the aliphatic ester of polyglycerol phosphate used in accordance with the present invention. Esterification: chemical reaction leading to the formation of an ester through the reaction of a fatty acid with an alcohol (in the present case polyglycerol) and the removal of water. Phosphorylation. Chemical reaction between phosphoric acid (or derivatives) and a compound, usually organic, with elimination of one molecule of water (for each radical of the acid introduced).

FIG. 2: Schematic representation of polyglycerol.

FIG. 3: Example of a fatty acid in its two isomeric forms cis and trans.

FIG. 4: Photographic images of the hair to be treated with evident presence of white hair.

FIG. 5: Photographic images of the hair treated with the dye.

FIG. 6: Photographic images of the final result.

### DETAILED DESCRIPTION OF THE INVENTION

For the purposes of the present invention, trichological composition (or trichological product) means a composition suitable for application to the hair or skin, intended for the hair or scalp care, hygiene and health.

Emulsifier means the set of ingredients necessary for the formation of a stable emulsion, formed for example by mixing the functional and accessory ingredients of a finished trichological product.

The aliphatic ester of polyglycerol phosphate used for the purposes of the invention is described as follows:
polyglycerol has a number of oligomeric units comprised between 4 and 20;
the phosphate ester is a polyglycerol monoester;
aliphatic ester is a polyglycerol ester with one or more fatty acid molecules of plant origin selected from the group consisting of saturated, monounsaturated and polyunsaturated fatty acids with carbon numbers comprised between 4 and 22 or their ester derivatives.

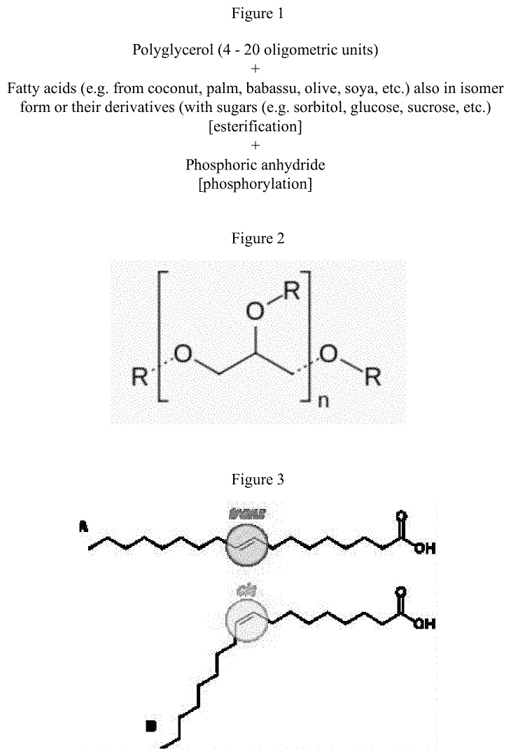

It should be noted that the polyglycerol phosphate aliphatic ester may be obtained by an esterification and phosphorylation reaction employing techniques known to the skilled person in the art and following the steps shown in FIG. 1.

Preferably, the polyglycerol phosphate aliphatic ester used for the purposes of the present invention is obtained by a process comprising the following steps:
Esterification between polyglycerol and fatty acid or trans-esterification between polyglycerol and triglyceride (vegetable oil).
Ester or trans-ester phosphorylation.

It should be noted that polyglycerol refers to an intermolecular glycerol ether formed by the condensation of 2 or more (n) glycerol molecules, with the elimination of a number (n-1) of water molecules (FIG. 2).

Known since the beginning of the century, polyglycerol esters began to be used in Europe and America in the 1940s. They were introduced to the Japanese market in the early 1960s and some of them were approved in the food sector as from 1981.

Polyglycerols can be synthesised in various ways by methods involving the condensation of glycerol by alkaline catalysis and the removal of water. The result of this synthesis is normally a mixture of oligomers, including unreacted glycerol, cyclic products and very high oligomers such as decaglycerol.

The polyglycerol used for the purposes of the present invention is thus a mixture of oligomers having a number of units varying between 4 and 20, preferably between 4 and 10, preferably between 4 and 6. It should be noted that in mixtures containing oligomers having a number of units greater than 4, traces of glycerol and polyglycerols having a number of oligomers less than 4 are still present.

A brief outline of polyglycerol chemistry can be given by looking at the following table where the linear oligomers of glycerol are described.

In polyglycerols, the number of OH groups corresponds to the degree of oligomerisation plus 2; therefore, diglycerol (n=2) has 4 OH groups and triglycerol (n=3) 5 OH groups etc. Of the two OH (primary and secondary) groups, it is the number of secondary OH groups that indicates the degree of oligomerisation.

| | (n) | OH groups | Primary | Secondary |
|---|---|---|---|---|
| Glycerol | 1 | 3 | 2 | 1 |
| Diglycerol | 2 | 4 | 2 | 2 |
| Triglycerol | 3 | 5 | 2 | 3 |
| Tetraglycerol | 4 | 6 | 2 | 4 |

It should be noted that, approximately, the molecular weight of the glycerol oligomers is between 166 g/mol (Linear Diglycerol) and 758 g/mol (Linear Decaglycerol).

According to a preferred embodiment, the polyglycerol phosphate esters are mixed mono- or polyester esters of saturated fatty acids, monounsaturated fatty acids and polyunsaturated fatty acids.

It should be noted that, according to a preferred form of embodiment, mixed polyglycerol polyesters may be obtained using mixtures of fatty acids (saturated, monounsaturated and/or polyunsaturated), for example in the form of vegetable oils.

Vegetable oils which may be used for the purposes of the invention include, for example, vegetable oils from coconut, palm, babassu, olive, soybean.

According to an alternative embodiment, polyglycerol esters are pure esters of saturated fatty acids or pure esters of monounsaturated fatty acids or pure esters of polyunsaturated fatty acids. A pure ester is defined as an ester in which the fatty acid esterifying one or more alcohol groups is of one kind only.

The polyglycerol usable for the purposes of the present invention may be a polyglycerol of plant or synthetic origin; by way of illustration, suitable polyglycerols for the purposes of the present invention are those marketed by Spiga Nord S.p.A.

Polyglycerol of vegetable origin means polyglycerol obtained by polycondensation of refined glycerins of vegetable origin chosen from at least one of the following: Vegetable origin glycerins, non-GMO (Genetically Modified Organisms) glycerins, Kosher glycerins (where Kosher means the certification issued for consumer products, indicating that the raw materials contained therein comply with the set of religious rules governing the nutrition of observant Jews) and Halal glycerins (where Halal means the certification issued for consumer products, indicating that the raw materials contained therein comply with the precepts of the Islamic Creed and Sharia′a; Halal products do not contain, for example, derivatives of animal origin or ethyl alcohol).

Plant origin means a compound obtained directly from a plant source or a synthetic compound with a chemical structure that imitates the molecules naturally present in the reference plant species.

It should be noted that polyglycerol can be esterified either with fatty acids of vegetable origin or with fatty acids of synthetic origin.

Such fatty acids are, preferably, aliphatic monocarboxylic fatty acids, which can also be used in isomer form.

According to a preferred embodiment, the saturated fatty acids have a number of carbon atoms comprised between 4 and 22.

Preferably, the saturated fatty acids suitable for the purposes of the invention are chosen from the group consisting of: butyric acid (C4:0), caprylic acid (C8:0), capric acid (C10:0), lauric acid (C12:0), myristic acid (C14:0), palmitic acid (C16:0), stearic acid (C18:0) and arachic acid (C20:0).

According to a preferred embodiment, the monounsaturated fatty acids have a number of carbon atoms comprised between 16 and 18.

Preferably, the monounsaturated fatty acids suitable for the purposes of the invention are chosen from the group consisting of: palmitoleic acid (C16:1), oleic acid (C18:1) and elaidinic acid (or trans-oleic acid) (C18:1).

According to a preferred embodiment, the polyunsaturated fatty acids have a number of carbon atoms comprised between 18 and 22 and a number of unsaturations comprised between 2 and 6.

Preferably, the polyunsaturated fatty acids suitable for the purposes of the invention are chosen from the group consisting of: linoleic acid (C18:2), alpha linoleic acid

(C18:3), arachic acid (C20:4), eicosapentaenoic acid (or EPA) (C20:5) and docosahexanoic acid (DHA) (C22:6).

According to a preferred form of implementation, the fatty acids are mixtures of the saturated, unsaturated and polyunsaturated fatty acids described above.

According to a preferred form of implementation, fatty acids suitable for esterification or trans-esterification of polyglycerol are chosen from one or more of the following:

| | |
|---|---|
| Butyric | C4:0 |
| Caprylic | C8:0 |
| Capric | C10:0 |
| Lauric | C12:0 |
| Myristic | C14:0 |
| Palmitic | C16:0 |
| Stearic | C18:0 |
| Arachic | C20:0 |
| Palmitoleic | C16:1 n-7 |
| Oleic | C18:1 n-9 (cis) |
| Elaidinic | C18:1 n-9 (trans) |

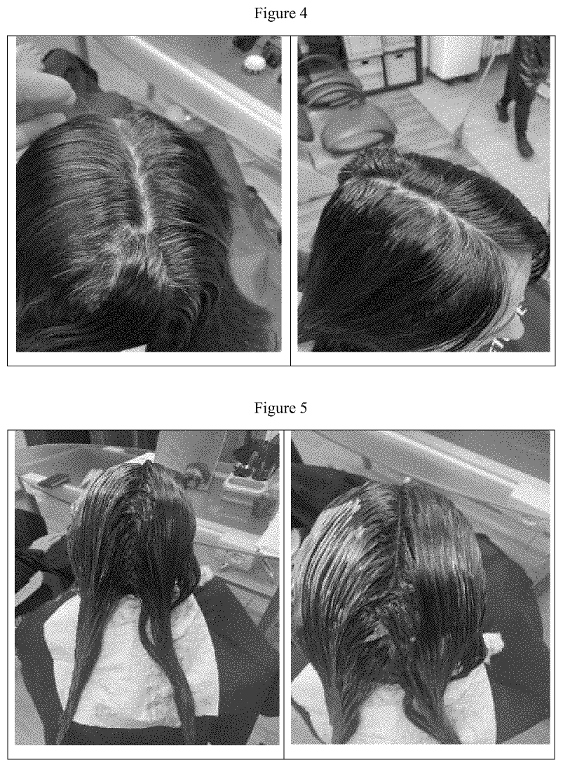

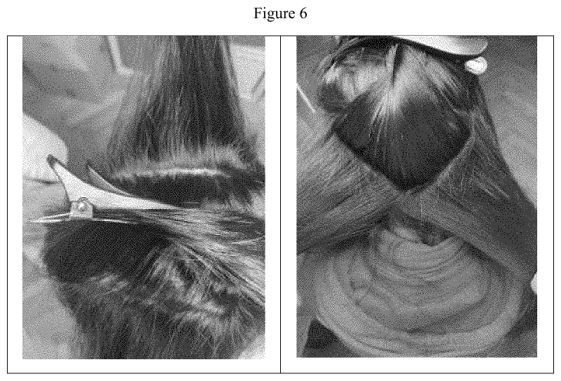

or from their homologous (cis) isomers which, having a more linear chain, advantageously have a lower melting point (FIG. 3).

Preferably, the phosphate ester between polyglycerol and stearic acid gave the best results for the purposes of the present invention.

According to an alternative embodiment, aliphatic polyglycerol esters may be formed from ester derivatives of the above-mentioned plant-derived fatty acids. Preferably, such ester derivatives are chosen from fatty acid esters with alditols and fatty acid esters with sugars.

Such fatty acid esters with alditols (alditol esters) or with sugars (sucresters) may be mono-, di- or tri-esters, preferably mono-esters.

Preferably, sugars suitable for the purposes of the invention are sucrose, glucose, fructose, lactose and maltose.

Preferably, alditols suitable for the purposes of the invention are sorbitol, mannitol and maltitol.

In the case of alditols with which fatty acids can be esterified prior to esterification with polyglycerol and subsequent phosphorylation, ester derivatives of fatty acids with sorbitol (sorbitan esters) have proved to be the most suitable for use.

According to an alternative embodiment, the aliphatic ester of polyglycerol phosphate is used in combination with other emulsifying agents to produce a stable emulsion useful in the preparation of a product for trichological use of interest.

Preferably, such further emulsifying agents are chosen from one or more of the following:
fatty alcohols having a number of carbon atoms comprised between 12 and 22 or their esters with fatty acids (e.g. cetyl stearate, cetyl palmitate)
anionic emulsifiers (e.g. sulphated alkyl ethers)
fatty acid glycerides (e.g. glyceryl stearates, glyceryl oleates).

It should be noted that, preferably, the aliphatic ester of polyglycerol phosphate has an HLB (Hydrophilic-lipophilic balance) value ranging between 5 and 12.

The polyglycerol phosphate aliphatic ester, when used in a mixture with other emulsifying agents, preferably constitutes 20-50% by weight of the total weight of the emulsifying agents used, preferably 30-50% by weight.

Polyglycerol phosphate aliphatic ester may be used as an emulsifier for the preparation of products for trichological use (semi-finished or finished products) in combination with active ingredients, excipients and/or diluents.

It should be noted that the active ingredients, excipients and diluents compatible with the aliphatic polyglycerol phosphate ester in the scope of the invention can be chosen from those known to the technician in the field, suitable for application to the hair and scalp, in order to customize and specialize the trichological product from a functional, technological or aesthetic point of view.

According to a preferred embodiment, the trichological product comprises polyglycerol phosphate ester, or a mixture thereof with other emulsifying excipients, in an amount between 3% and 40% by weight of the total weight of the trichological product (w/w), preferably between 5% and 40% (w/w), preferably between 10% and 40% (w/w), preferably between 10% and 35% (w/w).

Preferably, the trichological product, prepared using the polyglycerol phosphate aliphatic ester, is chosen from the group consisting of hair dyes, hair care and wellness products, hair conditioning and hair finishing products.

It should be noted that, in the case of hair dyes, the concentration of the polyglycerol phosphate ester varies between 5% and 10% by weight of the total weight of the dye.

Hair care and wellness products means masks, conditioners and creams applied to the hair or scalp to obtain a cosmetic, perfecting, volumising, polishing and restructuring effect; hair conditioning and finishing products are products to facilitate hair styling, holding and setting.

Examples of hair care and beauty products are masks for conditioning the hair, reconstructing and finishing the hair, masks with sebum-regulating and astringent functions, smoothing, curling, anti-frizz functions, etc.

Preferably, the trichological product is a hair dye.

For the purposes of the invention, a hair dye is understood to be a permanent hair dye, a temporary hair dye or a semi-permanent hair dye; according to a preferred form, decolorizers and oxidizers are also included in the definition of hair dye.

Preferably, the trichological product is a permanent hair dye.

### EXAMPLES

For illustrative purposes and without limitation, the following are examples of products for trichological use according to the present invention.

During colouring and decolouring treatments, the percentage of keratin present in the hair decreases, disulfide bridges and cysteine filaments break down. In order to restore brittle and damaged hair to its optimal condition, a shock treatment (reconstruction) and a maintenance treatment (mask, conditioner) are frequently used to preserve the normal structure of the hair after washing or as an “additive” during colouring, decolouring or other processes.

Hair conditioning mask

| Function | Ingredient | Concentration |
|---|---|---|
| Emulsifier | Polyglycerol/fatty acid | 3-5% |
| composition | phosphate | |
| | Cetylstearyl alcohol | 2-4% |
| | Glycerylmonostearate | 1-2% |
| Secondary | Propanediol (vegetable) | 3-5% |
| solvent | | |
| Surfactant | Non-ionic, amphoteric (APG, | 3-5% |
| | AAPB) | |
| Conditioning | Benamidopropyl dimethylamine | 2-5% |
| agents | | |
| Polishing | Natural oils and derivatives | 1-5% |
| agents | (without silicones) | |
| Speciality | Active ingredients and | 1-5% |
| products | functional substances | |
| Restructuring | Polymers, cationic copolymers | 1-3% |
| agents | | |
| Base solvent | Water | As needed at 100 |
| Fragrance | Perfume | As needed |

Hair restructuring cream (reconstruction)

| Function | Ingredient | Concentration |
|---|---|---|
| Emulsifier | Cetyl cystearyl alcohol | 5% |
| composition | Polyglycerol stearate | 4% |
| | phosphate | |
| | Sorbitan monostearate | 1% |
| Active | Keratin, chitin, cysteine etc. | 5% |
| substances | | |
| Functional | Nutrients, moisturisers, | 5% |
| substances | plasticisers | |
| Conditioners | Quaternary, cationic | 4-6% |
| | homopolymers | |
| Specialising | Copolymer amino acids and | 4% |
| substances | silanes | |
| Antioxidant | Sodium hydrosulphite | 0.5% |
| Chelating agent | Sequestrant | 0.1% |
| Basic solvent | Water, ethyl alcohol | As needed at 100 |
| Fragrance | Perfume | As needed |

All oxidising colouring products consist of two components: the first alkaline component, the dye, containing the colour precursors, and the second component, the oxidising emulsion (detector). A few minutes before application to the hair, the two components are mixed together, triggering a chemical reaction that creates the desired colour shade.

In the hair colouring process, hydrogen peroxide is therefore used as a means of decolouration or oxidation. It is used in emulsified form because, once combined with the dye, it must form a homogeneous compound with a viscosity that favours contact and adhesion to the hair, avoiding dripping on the face and neck. The possibility of producing emulsified hydrogen peroxide in a “green” version makes it possible to carry out an entire colouring process without polyethylene glycol derivatives.

Emulsified hydrogen peroxide

| | | Concentration | | |
|---|---|---|---|---|
| Function | Ingredient | 12 vol. | 20 vol. | 40 vol. |
| Emulsifier | Cetylstearyl alcohol | 2% | 2% | 2.5% |
| composition | Polyglycerol/fatty | 1.5% | 2% | 2.0% |
| | acid phosphate | | | |
| | Lauryl alcohol | 0.5% | 0.5% | 1.0% |
| Oxidant | Hydrogen peroxide | 10% | 18% | 34% |
| | (H2O2) 36%. | | | |
| Stabilisers | Oxyquiniline, Na | 0.5% | 0.5% | 0.5% |
| | pyrophosphate etc. | | | |
| pH corrector | Phosphoric acid | 0.5% | 0.5% | 0.5% |
| | solution etc. | | | |
| Water | Water | 85% | 76% | 59% |

### Effectiveness Tests

As proof of the validity of the emulsifying composition based on polyglycerol phosphate, we present a photographic comparison of the results obtained with the same hair dye formulated both with a traditional type of emulsifying composition containing ethoxylates (comparison example), and with a “Peg free” emulsifying composition (free of polyethylene glycols) according to the teachings in the patent.

The two dyes were formulated with an equal amount of emulsifying composition, were made by the same preparation method and contain the same amount of dyes, ingredients and excipients.

The effectiveness of the new solution was tested with the black dye (shade 1.0), a colour that is more difficult to prepare and stabilise than dyes of other colours due to the chemical nature of the dyes and their high concentration. In addition, an incorrectly made dark dye clearly shows defects in coverage and colour uniformity.

The experimentation was carried out by applying the two products using the same method to hair divided into two parts: on the left the traditional dye, on the right the innovative dye.

Hair dye comprising polyoxyethylene
emulsifiers (COMPARISON EXAMPLE)

| Function | Ingredient | Concentration |
|---|---|---|
| Emulsifier | Cetylstearyl alcohol | 15% |
| composition | Cetyltistearyl alcohol (20)OE | 8% |
| | Cetylstearyl alcohol (3)OE | 4% |
| | Glyceryl monostearate | 3% |
| Primary dye | Toluene-2,3-diamine sulphate | 4% |
| Secondary dye | Pigments, resorcinol | 4% |
| Plus | Special substances (oils, | max. 5% |
| | emollients, active ingredients) | |
| Alkalinising | Ammonia, amines etc. | max. 6% |
| Antioxidant | Sodium hydrosulphite | 0.2% |
| Chelating | Sequestrant | 0.1% |
| Basic Solvent | Water | 40-50% |
| Fragrance | Perfume | As needed |
| Surfactant | Amphoteric, non-ionic or | max. 5% |
| | anionic | |
| Secondary | Glycols or other | max. 5% |
| solvent | | |

Hair dye comprising a polyglycerol phosphate
aliphatic ester as emulsifier

| Function | Ingredient | Concentration |
|---|---|---|
| Emulsifier | Cetyl stearyl alcohol | 15% |
| composition | Polyglycerol stearate | 12% |
| | phosphate | |
| | Glyceryl monostearate | 3% |
| Primary dye | Toluene-2,3-diamine | 4% |
| | sulphate | |
| Secondary dye | Pigments, resorcinol | 4% |
| Plus | Special and functional | max. 5% |
| | substances (oils, emollients, | |
| | active ingredients) | |
| Alkalinising | Ammonia, amines etc. | max. 6% |
| Antioxidant | Sodium hydrosulphite | 0.2% |
| Chelating | Sequestrant | 0.1% |
| Basic solvent | Water | 40-50% |
| Fragrance | Perfume | As needed |
| Surfactant | Amphoteric, non-ionic | max. 5% |
| | or anionic | |
| Secondary | Propanediol or other | max. 5% |
| solvent | | |

Preparation method: The method for preparing any of the emulsifying compositions listed above is always the same:

The aqueous and fatty phases are brought to the same temperature, so that the fatty phase is completely dissolved.

Subsequently, the aqueous and fatty phases are united by mechanical action, ensuring that all hydrophilic substances are first dissolved in water and then all hydrophobic substances are dissolved in the fatty phase (with the exception of heat-sensitive components, which can be added at the end of the emulsion when the temperature falls below the critical level).

Once a good dispersion of one phase in the other is obtained, proceed with the gradual cooling, until the desired consistency and appearance is obtained.

1. Use of an aliphatic ester of a polyglycerol phosphate as an emulsifier for the preparation of compositions for trichological use, wherein,
the polyglycerol has a number of monomeric units ranging between 4 and 20;
the phosphate ester is a polyglycerol monoester;
the aliphatic ester is a polyglycerol ester with one or more fatty acids molecules, selected from the group consisting of saturated, mono-unsaturated and polyunsaturated fatty acids having a number of carbon atoms comprised between 4 and 22, or their ester derivatives.

2. Use according to claim 1 wherein the polyglycerol has a number of oligomeric units ranging between 4 and 10.

3. Use according to claim 1 wherein the fatty acids of the aliphatic ester are monocarboxylic fatty acids.

4. Use according to claim 1, wherein the fatty acids of the aliphatic ester are selected from the group consisting of: butyric acid (C4:0), caprylic acid (C8:0), capric acid (C10:0), lauric acid (C12:0), myristic acid (C14:0), palmitic acid (C16:0), stearic acid (C18:0), arachic acid (C20:0); palmitoleic acid (C16:1), oleic acid (C18:1); elaidinic acid (or trans-oleic acid) (C18:1); linoleic acid (C18:2), alpha linoleic acid (C18:3), arachic acid (C20:4), eicosapentaenoic acid (or EPA) (C20:5); docosahexaenoic acid (DHA) (C22:6) and combinations thereof.

5. Use according to claim 1, wherein the fatty acids ester derivatives are chosen from alditol or sucrose esters.

6. Use according to claim 1, wherein the aliphatic ester of the polyglycerol phosphate is used for the preparation of compositions for trichological use in combination with additional emulsifying agents selected from one or more of the following:
fatty alcohols having a number of carbon atoms comprised between 12 and 22 or their esters with fatty acids;
anionic emulsifiers;
glycerides of fatty acids.

7. Use according to claim 6, wherein the aliphatic ester of the polyglycerol phosphate constitutes from 30% to 50% of the total emulsifying agents used in the preparation of a composition for trichological use.

8. Use according to claim 1, wherein the aliphatic ester of the polyglycerol phosphate is used in concentration comprised between 3% and 40% by weight of the total weight of a composition for trichological use (w/w).

9. Use according to claim 1, wherein the trichological product compositions for trichological use are selected from the group consisting of: hair dyes, hair care and wellness products, hair conditioners and hairdressing products.
